(19) United States
(12) Patent Application Publication
(10) Pub. No.: US 2007/0021329 A1
(43) Pub. Date: Jan. 25, 2007

(54) Treatment of hypersensitivity conditions

(75) Inventors: Ian Alexander Shiels, Muirlea (AU); Stephen Maxwell Taylor, Bellbird Park (AU)

Correspondence Address: HAYNES AND BOONE, LLP, 901 MAIN STREET, SUITE 3100, DALLAS, TX, 75202, US

(73) Assignee: THE University of QUEENSLAND, Brisbane (AU)

(21) Appl. No.: 10/531,565

(22) PCT Filed: Oct. 16, 2003
(86) PCT No.: PCT/AU03/01374

§ 371 Date: Aug. 11, 2005

(30) Foreign Application Priority Data
Oct. 17, 2002 (AU) 2002952129

(51) Int. Cl. A61K 38/12

(52) U.S. Cl. 514/9

(57) ABSTRACT

This invention relates to methods of treatment of hypersensitivity conditions such as asthma and other allergic conditions, and especially to treatment of these conditions with cyclic peptidic and peptidomimetic compounds which have the ability to modulate the activity of G protein-coupled receptors. The compounds preferably act as antagonists of the C5a receptor, and are active against C5a receptors on polymorphonuclear leukocytes and macrophages. Particularly preferred compounds for use in the methods of the invention are disclosed.

### FIELD OF THE INVENTION

This invention relates to the treatment of hypersensitivity conditions such as asthma and other allergic conditions, and especially to treatment of these conditions with novel cyclic peptidic and peptidomimetic compounds which have the ability to modulate the activity of G protein-coupled receptors. The compounds preferably act as antagonists of the C5a receptor, and are active against C5a receptors on polymorphonuclear leukocytes and macrophages.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

G protein-coupled receptors are prevalent throughout the human body, comprising approximately 60% of known cellular receptor types, and mediate signal transduction across the cell membrane for a very wide range of endogenous ligands. They participate in a diverse array of physiological and pathophysiological processes, including, but not limited to those associated with cardiovascular, central and peripheral nervous system, reproductive, metabolic, digestive, immunological, inflammatory, and growth disorders, as well as other cell-regulatory and proliferative disorders. Agents which selectively modulate functions of G protein-coupled receptors have important therapeutic applications. These receptors are becoming increasingly recognised as important drug targets, due to their crucial roles in signal transduction (G protein-coupled Receptors, IBC Biomedical Library Series, 1996).

One of the most intensively studied G protein-coupled receptors is the receptor for C5a. C5a is one of the most potent chemotactic agents known, and recruits neutrophils and macrophages to sites of injury; alters their morphology; induces degranulation; increases calcium mobilisation, vascular permeability (oedema) and neutrophil adhesiveness; contracts smooth muscle; stimulates release of inflammatory mediators, including histamine, TNF-α, IL-1, IL-6, IL-8, prostaglandins, and leukotrienes, and of lysosomal enzymes; promotes formation of oxygen radicals; and enhances antibody production (Gerard and Gerard, 1994).

Agents which limit the pro-inflammatory actions of C5a have potential for inhibiting chronic inflammation, and its accompanying pain and tissue damage. For these reasons, molecules which prevent C5a from binding to its receptors are useful for treating chronic inflammatory disorders driven by complement activation. Such compounds also provide valuable new insights into the mechanisms of complement-mediated immunity.

In our previous application No. PCT/AU98/00490 we described the three-dimensional structure of some analogues of the C-terminus of human C5a, and used this information to design novel compounds which bind to the human C5a receptor (C5aR), behaving as either agonists or antagonists of C5a. It had previously been thought that a putative antagonist might require both a C-terminal arginine and a C-terminal carboxylate for receptor binding and antagonist activity (Konteatis et al, 1994). We showed that in fact a terminal carboxylate group is not generally required either for high affinity binding to

C5aR or for antagonist activity. Instead we found that a hitherto unrecognised structural feature, a turn conformation, was the key recognition feature for high affinity binding to the human C5a receptor on neutrophils. As described in our international patent application No. PCT/AU02/01427, filed on 17^{th} Oct. 2002, we used further refinements of these findings to design more tightly constrained structural templates which enable hydrophobic groups to be assembled into a hydrophobic array for interaction with a C5a receptor. We have subsequently found that a preferred compound of this class is able to inhibit cardiac and pulmonary fibrosis, and this is described in our international patent application No. PCT/AU03/00415, filed on 7^{th} Apr. 2003. The entire disclosures of these specifications are incorporated herein by this reference.

Asthma is a potentially life-threatening condition characterized by paroxysmal attacks of bronchospasm, which cause wheezing, tightness in the chest, and difficulty in breathing. Asthmatic attacks may be provoked by exposure to a variety of stimuli, such as allergens, infection, exercise, changes in ambient temperature or humidity, cigarette smoke, stress or emotional upset. Although attacks can occur at any stage of life, asthma usually has its onset during childhood; it may be associated with other hypersensitivity conditions such as eczema or hay fever. Asthma affects up to 16 million Americans, including approximately 10-12% of children under age 18, and its incidence is increasing. It is more common in individuals under the age of 40, and is the leading cause of absence from school and admission to hospital among children. People who have a family history of asthma or who have allergies have an increased risk of developing the disease.

Treatment is predominantly with bronchodilators such as β₂-adrenergic agonists, administered orally or as inhaled aerosols, and corticosteroids are used in severe cases. Metered-dose inhalers, dry powder inhalers and nebulizers are widely used for inhalation therapy. However, despite intensive research the available therapies are unable to control symptoms in all patients. Asthma is associated with very significant morbidity, is responsible for a high proportion of hospital emergency room admissions, and causes a number of deaths each year. In addition to this, the economic cost of asthma is very high.

Eczema or eczematous dermatitis is an inflammatory dermatitis associated with itching and vesicle formation, followed by weeping and crusting of the lesions. In the more chronic forms there may be lichenification and/or thickening, excoriation, or pigmentation changes; these can be disfiguring. The allergic or atopic form of eczema can be caused by a variety of allergens, which may be inhaled, contact or food allergens; it can be very difficult to identify the allergen responsible. Atopic eczema is common in childhood. Topical steroids are the primary first-line treatment. Hand dermatitis is a form of atopic dermatitis, which affects an estimated 1.9 million people in the United States. Chronic hand dermatitis will repeatedly relapse or flare. It can involve 25-90% of the hands and affect one or both surfaces. Treatment is usually with topical corticosteroids, but this may have limited success, and is associated with side effects such as skin atrophy. Targretin, a vitamin A analogue, has recently been approved in the United States for treatment of hand dermatitis.

Dermatitis is also a major veterinary problem, and is common in domestic animals such as horses, and in companion animals such as cats and dogs. For example, approximately 5% to 10% of the current U.S. dog population, or four to seven million dogs, is affected. Due to the chronic nature of allergic dermatitis in affected dogs, the estimated number of treatments per year is in excess of 8 million. Current treatment primarily uses medicated shampoos and conditioners with severe cases requiring treatment with glucocorticoids.

A variety of agents, including immunoglobulin E (IgE)-targeting monoclonal antibodies, tumour necrosis factor antagonists, immunosuppressive agent such as tacrolimus and pimecrolimus, and phototherapies, are in various stages of clinical trial for the treatment of hypersensitivity conditions.

However, there is a great need in the art for effective, non-toxic agents for the treatment of asthma and other hypersensitivity conditions, which do not require administration by injection, and which can be produced at reasonable cost. To our knowledge none of these approved or experimental agents, and in particular no small molecule agent, targets the C5a receptor.

### SUMMARY OF THE INVENTION

We now show for the first time that a specific inhibitor of the C5a receptor is able to ameliorate asthma in a mammal. This is the first reported case of an inhibitor of the complement system being used to modulate pathology in asthma.

According to a first aspect, the invention provides a method of treatment of a hypersensitivity conditions, comprising the step of administering an effective amount of an inhibitor of a G protein-coupled receptor to a subject in need of such treatment.

Preferably the inhibitor is a compound which
(a) is an antagonist of a G protein-coupled receptor,
(b) has substantially no agonist activity, and
(c) is a cyclic peptide or peptidomimetic compound of formula I

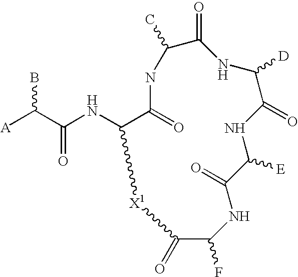

where A is H, alkyl, aryl, NH₂, NH-alkyl, N(alkyl)₂, NH-aryl, NH-acyl, NH-benzoyl, NHSO₃, NHSO₂-alkyl, NHSO₂-aryl, OH, O-alkyl, or O-aryl;

B is an alkyl, aryl, phenyl, benzyl, naphthyl or indole group, or the side chain of a D- or L-amino acid such as L-phenylalanine or L-phenylglycine, but is not the side chain of glycine, D-phenylalanine, L-homophenylalanine, L-tryptophan, L-homotryptophan, L-tyrosine, or L-homotyrosine;

C is a small substituent, such as the side chain of a D-, L- or homo-amino acid such as glycine, alanine, leucine, valine, proline, hydroxyproline, or thioproline, but is preferably not a bulky substituent such as isoleucine, phenylalanine, or cyclohexylalanine;

D is the side chain of a neutral D-amino acid such as D-Leucine, D-homoleucine, D-cyclohexylalanine, D-homocyclohexylalanine, D-valine, D-norleucine, D-homo-norleucine, D-phenylalanine, D-tetrahydroisoquinoline, D-glutamine, D-glutamate, or D-tyrosine, but is preferably not a small substituent such as the side chain of glycine or D-alanine, a bulky planar side chain such as D-tryptophan, or a bulky charged side chain such as D-arginine or D-Lysine;

E is a bulky substituent, such as the side chain of an amino acid selected from the group consisting of L-phenylalanine, L-tryptophan and L-homotryptophan, or is L-1-napthyl or L-3-benzothienyl alanine, but is not the side chain of D-tryptophan, L-N-methyltryptophan, L-homophenylalanine, L-2-naphthyl L-tetrahydroisoquinoline, L-cyclohexylalanine, D-leucine, L-fluorenylalanine, or L-histidine;

F is the side chain of L-arginine, L-homoarginine, L-citrulline, or L-canavanine, or a bioisostere thereof, ie. a side chain in which the terminal guanidine or urea group is retained, but the carbon backbone is replaced by a group which has different structure but is such that the side chain as a whole reacts with the target protein in the same way as the parent group; and

X is —(CH₂)ₙNH— or (CH₂)ₙ—S—, where n is an integer of from 1 to 4, preferably 2 or 3; —(CH₂)₂O—; —(CH₂)₃O—; —(CH₂)₃—; —(CH₂)₄—; —CH₂COCHRNH—; or —CH₂—CHCOCHRNH—, where R is the side chain of any common or uncommon amino acid.

In C, both the cis and trans forms of hydroxyproline and thioproline may be used.

Preferably A is an acetamide group, an aminomethyl group, or a substituted or unsubstituted sulphonamide group.

Preferably where A is a substituted sulphonamide, the substituent is an alkyl chain of 1 to 6, preferably 1 to 4 carbon atoms, or a phenyl or toluyl group.

In a particularly preferred embodiment, the compound has antagonist activity against C5aR, and has no C5a agonist activity.

The compound is preferably an antagonist of C5a receptors on human and mammalian cells including, but not limited to, human polymorphonuclear leukocytes and human macrophages. The compound preferably binds potently and selectively to C5a receptors, and more preferably has potent antagonist activity at sub-micromolar concentrations. Even more preferably the compound has a receptor affinity IC50<25 μM, and an antagonist potency IC50<1 μm.

Most preferably the compound is selected from the group consisting of compounds 1 to 6, 10 to 15, 17, 19, 20, 22, 25, 26, 28, 30, 31, 33 to 37, 39 to 45, 47 to 50, 52 to 58 and 60 to 70 described in provisional application No. PCT/AU02/01427. In a particularly preferred embodiment, the compound is PMX53 (compound 1), compound 33, compound 60 or compound 45 described therein.

In a second aspect the invention provides the use of a compound as defined above in the manufacture of a medicament for the treatment of a hypersensitivity condition.

The hypersensitivity condition may be any state in which complement-mediated tissue damage results from the immune reaction of a sensitised or immunized individual to a subsequent exposure to antigen. These include but are not limited to Type II immediate hypersensitivity (cytotoxic) and Type III (complex-mediated) immediate hypersensitivity, such as asthma, eczema or dermatitis, and Arthus-type reactions such as serum sickness, glomerulonephritis, hypereosinophilia syndrome, and farmer's lung. In one preferred embodiment the hypersensitivity condition is asthma, eczema or dermatitis.

The inhibitor may be used in conjunction with one or more other agents for the treatment of hypersensitivity conditions. For asthma, these include but are not limited to bronchodilators such as β₂-adrenergic agonists, including but not limited to albuterol (Ventolin), anti-histamines such as chlorpheniramine, diphenhydramine, or mepyramine, mast cell stabilisers like nedocromil, leukotriene blockers like zileutin, montelukast and zaphirlukast, muscarinic antagonists like ipratropium and corticosteroids such as prednisolone, budesonide and fluticasone and synthetic steroids or analogues thereof. For eczema these include but are not limited to topical corticosteroids and synthetic steroids or analogues thereof, and Vitamin A analogues such as bexarotene (“Targretin”). The inhibitor may alternatively or additionally be used in conjunction with antiinflammatories, such as eicosapentanoic acid derivatives and omega-3 oils.

The compounds of the invention may be formulated for oral, parenteral, inhalational, intranasal, topical or transdermal use. Suitable formulations for administration by any desired route may be prepared by standard methods, for example by reference to well-known textbooks such as Remington: The Science and Practice of Pharmacy, Vol. II, 2000 (20^{th} edition), A. R. Gennaro (ed), Williams & Wilkins, Pa.

While the invention is not in any way restricted to the treatment of any particular animal or species, it is particularly contemplated that the method of the invention will be useful in medical treatment of humans, and will also be useful in veterinary treatment, particularly of companion animals such as cats and dogs, livestock such as cattle, horses and sheep, and zoo animals, including non-human primates, large bovids, felids, ungulates and canids.

The compound may be administered at any suitable dose and by any suitable route. Oral, topical or intranasal administration is preferred, because of the greater convenience and acceptability of these routes. Oral formulations are particularly preferred. It is expected that most if not all compounds of the invention will be stable in the presence of metabolic enzymes, such as those of the gut, blood, lung or intracellular enzymes. Such stability can readily be tested by routine methods known to those skilled in the art.

The effective dose will depend on the nature of the condition to be treated, and the age, weight, and underlying state of health of the individual treatment. This will be at the discretion of the attending physician or veterinarian. Suitable dosage levels may readily be determined by trial and error experimentation, using methods which are well known in the art.

### BRIEF DESCRIPTION OF THE FIGURES

FIG. 1 shows the inhibition of the vascular leakage associated with a dermal Arthus reaction by intravenous (A), oral (B) and topical (C) AcF-[OPdChaWR], and appropriate controls (D).

FIG. 2 shows the inhibition of the rise in circulating TNFα associated with a dermal Arthus reaction by intravenous (A), oral (B) and topical (C) AcF-[OPdChaWR], and appropriate topical controls (D).

FIG. 3 shows the reduction of the pathology index associated with a dermal Arthus reaction by intravenous, oral and topical AcF-[OPdChaWR].

FIG. 4 illustrates the response of a dog with allergic dermatitis associated with flea infestation accompanied by demodectic mange to treatment with PMX53.

### DETAILED DESCRIPTION OF THE INVENTION

It is to be clearly understood that this invention is not limited to the particular materials and methods described herein, as these may vary. It is also to be understood that the terminology used herein is for the purpose of describing particular embodiments only, and it is not intended to limit the scope of the present invention, which will be limited only by the appended claims.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

As used herein, the singular forms “a”, “an”, and “the” include plural reference unless the context clearly dictates otherwise. Thus, for example, a reference to “an enzyme” includes a plurality of such enzymes, and a reference to “an amino acid” is a reference to one or more amino acids. Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are now described.

Abbreviations used herein are as follows:
D-Cha D-cyclohexylamine
LPS lipopolysaccharide
PMN polymorphonuclear granulocyte
RMSD root mean square deviation
rp-HPLC reverse phase-high performance liquid chromatography
TFA trifluoroacetic acid;

Throughout the specification conventional single-letter and three-letter codes are used to represent amino acids.

For the purposes of this specification, the term “alkyl” is to be taken to mean a straight, branched, or cyclic, substituted or unsubstituted alkyl chain of 1 to 6, preferably 1 to 4 carbons. Most preferably the alkyl group is a methyl group. The term “acyl” is to be taken to mean a substituted or unsubstituted acyl of 1 to 6, preferably 1 to 4 carbon atoms. Most preferably the acyl group is acetyl. The term “aryl” is to be understood to mean a substituted or unsubstituted homocyclic or heterocyclic aryl group, in which the ring preferably has 5 or 6 members.

A “common” amino acid is a L-amino acid selected from the group consisting of glycine, leucine, isoleucine, valine, alanine, phenylalanine, tyrosine, tryptophan, aspartate, asparagine, glutamate, glutamine, cysteine, methionine, arginine, lysine, proline, serine, threonine and histidine.

An “uncommon” amino acid includes, but is not restricted to, D-amino acids, homo-amino acids, N-alkyl amino acids, dehydroamino acids, aromatic amino acids other than phenylalanine, tyrosine and tryptophan, ortho-, meta- or para-aminobenzoic acid, ornithine, citrulline, canavanine, norleucine, γ-glutamic acid, aminobutyric acid, L-fluorenylalanine, L-3-benzothienylalanine, and α,α-disubstituted amino acids.

Generally, the terms “treating”, “treatment” and the like are used herein to mean affecting a subject, tissue or cell to obtain a desired pharmacological and/or physiological effect. The effect may be prophylactic in terms of completely or partially preventing a disease or sign or symptom thereof, and/or may be therapeutic in terms of a partial or complete cure of a disease.

“Treating” as used herein covers any treatment of, or prevention of disease in a vertebrate, a mammal, particularly a human, and includes: preventing the disease from occurring in a subject who may be predisposed to the disease, but has not yet been diagnosed as having it; inhibiting the disease, ie., arresting its development; or relieving or ameliorating the effects of the disease, ie., cause regression of the effects of the disease.

The invention includes the use of various pharmaceutical compositions useful for ameliorating disease. The pharmaceutical compositions according to one embodiment of the invention are prepared by bringing a compound of formula I, analogue, derivatives or salts thereof and one or more pharmaceutically-active agents or combinations of compound of formula I and one or more pharmaceutically-active agents into a form suitable for administration to a subject using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, eg. in Langer, Science, 249: 1527, (1990). Formulations for oral use may be in the form of hard gelatin capsules, in which the active ingredient is mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

Aqueous suspensions normally contain the active materials in admixture with excipients suitable for the manufacture of aqueous suspensions. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be (a) a naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono-or diglycerides. In addition, fatty acids such as oleic acid may be used in the preparation of injectables.

Compounds of formula I may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the compound of formula I of the present invention will usually be of the order of about 0.5 mg to about 20 mg per kilogram body weight, with a preferred dosage range between about 0.5 mg to about 10 mg per kilogram body weight per day (from about 0.5 g to about 3 g per patient per day). The amount of active ingredient which may be combined with the carrier materials to produce a single dosage will vary, depending upon the host to be treated and the particular mode of administration. For example, a formulation intended for oral administration to humans may contain about 5 mg to 1 g of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the invention may form solvates with water or common organic solvents. Such solvates are encompassed within the scope of the invention.

The compounds of the invention may additionally be combined with other therapeutic compounds to provide an operative combination. It is intended to include any chemically compatible combination of pharmaceutically-active agents, as long as the combination does not eliminate the activity of the compound of formula I of this invention.

In evaluation of the compounds of the invention, conventional measures of efficacy may be used. For example, for asthma commonly-used primary efficacy end-points include lung function tests such as spirometry or measurement of vital capacity, or self-monitoring using a peak flow meter. For eczema, evaluation of efficacy may be based on:
(a) Physician's Static Assessment (PSA), a primary endpoint required by the United States food and Drug Administration, which calls for 90% or greater improvement in signs and symptoms, is equivalent to a clear or almost clear condition on at least two observations 21 days apart, or
(b) Physician's Global Assessment (PGA), which calls for 50% or greater improvement.

The invention will now be described by way of reference only to the following general methods and experimental examples.

General Methods

Cyclic peptide compounds of formula I are prepared according to methods described in detail in our earlier applications No. PCT/AU98/00490 and PCT/AU02/01427. An alternative method of synthesis is described in our Australian provisional application No. 2003902743. The entire disclosures of these specifications are incorporated herein by this reference. While the invention is specifically illustrated with reference to the compound AcF-[OPdChaWR] (PMX53), whose corresponding linear peptide is Ac-Phe-Orn-Pro-dCha-Trp-Arg, it will be clearly understood that the invention is not limited to this compound.

Compounds 1-6, 17, 20, 28, 30, 31, 36 and 44 disclosed in International patent application No. PCT/AU98/00490 and compounds 10-12, 14, 15, 25, 33, 35, 40, 45, 48, 52, 58, 60, 66, and 68-70 disclosed for the first time in Australian provisional application No. PCT/AU02/01427 have appreciable antagonist potency (IC50<1 μM) against the C5a receptor on human neutrophils. PMX53 (compound 17 of PCT/AU98/00490; also identified as compound 1 in PCT/AU02/01427) and compounds 33, 45 and 60 of PCT/AU02/01427 are most preferred.

We have found that all of the compounds of formula I which have so far been tested have broadly similar pharmacological activities, although the physicochemical properties, potency, and bioavailability of the individual compounds vary somewhat, depending on the specific substituents.

The following general tests may be used for initial screening of candidate inhibitor of G protein-coupled receptors, and especially of C5a receptors.

Receptor-Binding Assay

Assays were performed with fresh human PMNs, isolated as previously described (Sanderson et al, 1995), using a buffer of 50 MM HEPES, 1 mM CaCl₂, 5 mM MgCl₂, 0.5% bovine serum albumin, 0.1% bacitracin and 100 μM phenylmethylsulfonyl fluoride (PMSF). In assays performed at 4° C., buffer, unlabelled human recombinant C5a (Sigma) or peptide, Hunter/Bolton labelled ¹²⁵I-C5a (˜20 pM) (New England Nuclear, Mass.) and PMNs (0.2×10⁶) were added sequentially to a Millipore Multiscreen assay plate (HV 0.45) having a final volume of 200 μL/well. After incubation for 60 min at 4° C., the samples were filtered and the plate washed once with buffer. Filters were dried, punched and counted in an LKB gamma counter. Non-specific binding was assessed by the inclusion of 1 mM peptide or 100 nM C5a, which typically resulted in 10-15% total binding.

Data was analysed using non-linear regression and statistics with Dunnett post-test.

Myeloperoxidase Release Assay for Antagonist Activity

Cells were isolated as previously described (Sanderson et al, 1995) and incubated with cytochalasin B (5 μg/mL, 15 min, 37° C.). Hank's Balanced Salt solution containing 0.15% gelatin and peptide was added on to a 96 well plate (total volume 100 μL/well), followed by 25 μL cells (4×10⁶/mL). To assess the capacity of each peptide to antagonise C5a, cells were incubated for 5 min at 37° C. with each peptide, followed by addition of C5a (100 nM) and further incubation for 5 min. Then 50 μL of sodium phosphate (0.1M, pH 6.8) was added to each well, the plate was cooled to room temperature, and 25 μL of a fresh mixture of equal volumes of dimethoxybenzidine (5.7 mg/mL) and H₂O₂ (0.51%) was added to each well. The reaction was stopped at 10 min by addition of 2% sodium azide. Absorbances were measured at 450 nm in a Bioscan 450 plate reader, corrected for control values (no peptide), and analysed by non-linear regression.

### EXAMPLE 1

### Reverse Passive Arthus Reaction in the Rat

A reverse passive peritoneal Arthus reaction was induced as previously described (Strachan et al., 2000), and a group of rats were pretreated prior to peritoneal deposition of antibody with AcF-[OPdChaWR] (1) by oral gavage (10 mg kg⁻¹ dissolved in 10% ethanol/90% saline solution to a final volume of 200 μl) or an appropriate oral vehicle control 30 min prior to deposition of antibody. Female Wistar rats (150-250 g) were anaesthetised with ketamine (80 mg kg⁻¹ i.p.) and xylazine (12 mg kg⁻¹ i.p.).

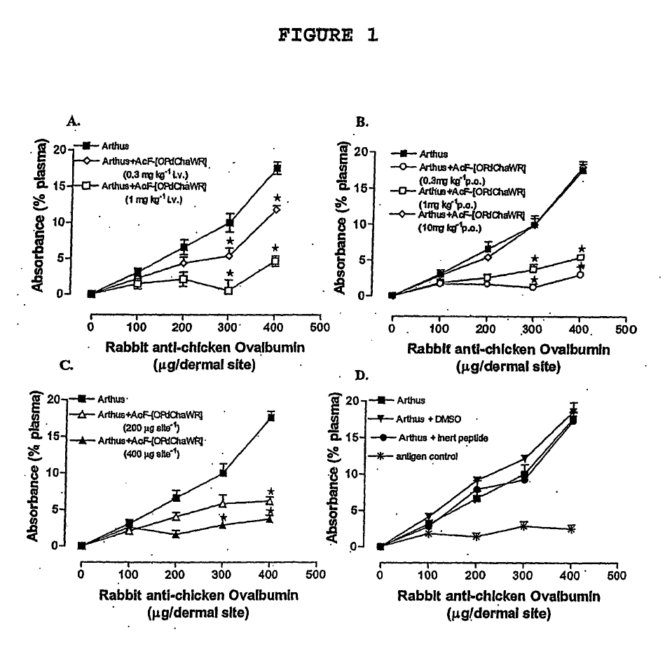

The lateral surfaces of the rat were carefully shaved and 5 distinct sites on each lateral surface clearly delineated. A reverse passive Arthus reaction was induced in each dermal site by injecting Evans blue (15 mg kg⁻¹ i.v.), chicken ovalbumin (20 mg kg⁻¹ i.v.) into the femoral vein 10 min prior to the injection of antibody. Rabbit anti-chicken ovalbumin (saline only, 100, 200, 300 or 400 μg antibody in a final injection volume of 30 μL) was injected in duplicate at two separate dermal sites on each lateral surface of the rat, giving a total of 10 injection sites per rat. Rats were placed on a heating pad, and anaesthetic was maintained over a 4 h-treatment period with periodic collection of blood samples. Blood was allowed to spontaneously clot on ice, and serum samples were collected and stored at −20° C. Four hours after induction of the dermal Arthus reaction, the anaesthetised rat was euthanased and a 10 mm² area of skin was collected from the site of each Arthus reaction. Skin samples were stored in 10% buffered formalin for at least 10 days before histological analysis using haematoxylin and eosin stain. Additionally, a second set of skin samples were placed in 1 mL of formamide overnight, and the absorbance of Evans blue extraction measured at 650 nm, as an indicator of serum leakage into the dermis. FIG. 1 shows the optical density of dermal punch extracts following intradermal injection of rabbit anti-chicken ovalbumin at 0-400 μg site⁻¹ following pretreatment with AcF-[OPdChaWR] intravenously, orally or topically. Data are shown as absorbance at 650nm as a percentage of the plasma absorbance, as mean values ±SEM (n=3-6). *indicates a P value ≦0.05 when compared to Arthus control values.

Rats were pretreated with the C5aR antagonist, AcF-[OPdChaWR] (1) as the TFA salt, either intravenously (0.3-1 mg kg⁻¹ in 200 μL saline containing 10% ethanol, 10 min prior to initiation of dermal Arthus), orally (0.3-10 mg kg⁻¹ in 200 μL saline containing 10% ethanol by oral gavage, 30 min prior to initiation of dermal Arthus in rats denied food access for the preceding 18 hours) or topically (200-400 μg site⁻¹ 10 min prior to initiation of dermal Arthus reaction), or with the appropriate vehicle control. Topical application of the antagonist involved application of 20 μl of a 10-20 mg mL⁻¹ solution in 10% dimethyl sulphoxide (DMSO), which was then smeared directly onto the skin at each site, 10 min prior to induction of the Arthus reaction.

The saline-only injection site from rats treated with Evans blue only served as antigen controls, the saline-only injection site from rats treated with Evans blue plus topical DMSO only served as a vehicle control, the saline-only injection site from rats treated with Evans blue plus either intravenous, oral or topical antagonist only served as antagonist controls, and Evans blue plus dermal rabbit anti-chicken ovalbumin served as antibody controls. Topical application of the peptide AcF-[OPGWR] which has similar chemical composition and solubility as AcF-[OPdChaWR] (1), but with an IC₅₀ binding affinity of >1 mM in isolated human PMNs, served as an inactive peptide control. AcF-[OPGWR] was also dissolved in 10% DMSO and applied topically at 400 μg site⁻¹ 10 min prior to initiation of the Arthus reaction.

TNFα Measurement

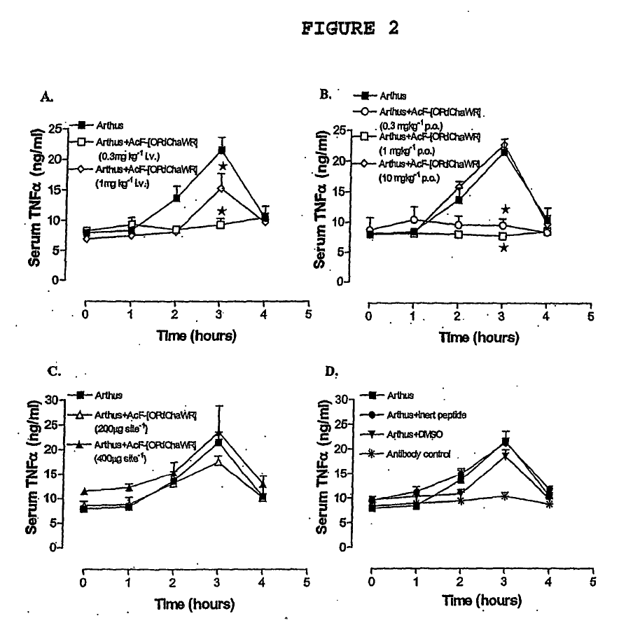

Serum TNFα concentrations were measured using an enzyme-linked immunosorbent assay (ELISA) kit (Strachan et al., 2000). Antibody pairs used were a rabbit anti-rat TNFα antibody coupled with a biotinylated murine anti-rat TNFα antibody. FIG. 2 shows the serum TNFα concentrations at regular intervals after initiation of a dermal Arthus reaction, with group of rats pretreated with AcF-[OPdChaWR] intravenously, orally or topically. Data are shown as mean values ±SEM (n=3-6). *indicates a P value of ≦0.05 when compared to Arthus control values.

Interleukin-6 Measurement

An ELISA method as described previously was used to measure serum and peritoneal lavage fluid interleukin-6 (IL-6) concentrations (Strachan et al., 2000).

Pathology Assessment

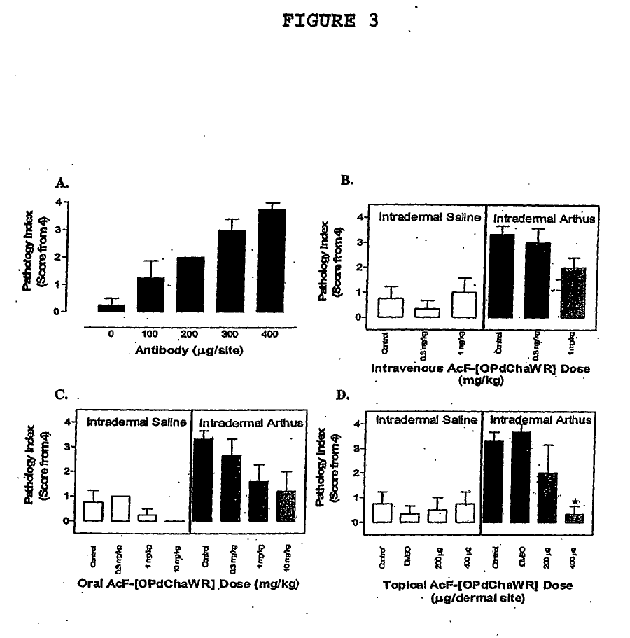

Rat skin samples were fixed in 10% buffered formalin for at least 10 days, and stained with haematoxylin and eosin using standard histological techniques. Dermal samples were analysed in a blind fashion for evidence of pathology, and the degree of rat PMN infiltration was scored on a scale of 0-4. Initiation of a dermal Arthus reaction resulted in an increase in interstitial neutrophils, which was quantified in the following manner. Sections were given a score of 0 if no abnormalities were detected. A score of 1 indicated the appearance of increased PMNs in blood vessels, but no migration of inflammatory cells out of the lumen. A score of 2 and 3 indicated the appearance of increasing numbers of PMNs in the interstitial tissue and more prominent accumulations of inflammatory cells around blood vessels. A maximal score of 4 indicated severe pathological abnormalities were present in dermal sections, with excessive infiltration of PMNs into the tissues and migration of these cells away from blood vessels. FIG. 3 shows that intradermal injection of increasing amounts of antibody leads to a dose-responsive increase in the pathology index scored by dermal samples (A). Data are shown for dermal samples intradermally injected with saline or 400 μg antibody per site (n=5) in rats pretreated with AcF-[OPdChaWR] intravenously (B) (n=3), orally (C) (n=3) and topically (D) (n=3). Data are shown as mean values ±SEM. * P≦0.05 when compared to Arthus values using a non-parametric t-test.

### EXAMPLE 2

### Treatment of Asthma in a Tiger Cub

Respiratory problems were first noticed in Kaasha, a female Bengal Tiger cub at the Dreamworld park, Australia, at the age of 10 weeks and around 10 kg body weight. The initial clinical signs observed were a mild to moderate increase in respiratory effort after feeding, followed within a few days by a continuous increase in respiratory effort. At no time prior to the initial clinical signs had Kaasha's keepers noticed any change in demeanour, appetite, activity level, or other parameter that might indicate illness. The rectal temperature was normal at the time of initial veterinary examination, and remained normal when measured over the following weeks. The principal clinical signs included increased respiratory effort characterised by a prolonged two-phase forced expiration, fine pulmonary crackles particularly in dorsal lobes, and a bronchointerstitial pattern and air entrapment on radiographs.

Initial treatment, pending definitive diagnosis, included antibiotics, Clavulox (7 mg/kg administered subcutaneously), doxycycline (5 mg/kg administered orally), and enrofloxacin (5 mg/kg administered subcutaneously), and terbutaline (0.3 mg/kg administered orally).

Two weeks after initial detection of clinical signs, Kaasha experienced an episode of severe dyspnoea with open-mouthed breathing. The episode lasted 20 to 30 seconds and resolved spontaneously.

Following repeated clinical examinations, radiography, clinical pathology (blood: CBC, MBA) (Blood biochemistry and haematology) and bronchoalveolar lavage cytology, a diagnosis of lower airway inflammation, of unknown aetiology, was made. Culture of bronchoalveolar lavage samples for microorganisms did not indicate the presence of any bacterial infection.

The anatomical diagnosis of neutrophil-dominated lower airway inflammation was confirmed histologically by a thoracoscopically-guided lung biopsy, although the pathologist also noted mixed inflammation of the interstitium. Further culture of the biopsy and polymerase chain reaction (PCR) for Feline viral rhinotracheitis virus and Feline Calicivirus and Chlamydia failed to provide convincing information regarding the aetiology of the condition.

Approximately three weeks after initial clinical signs, the treatment included Clavulox (7 mg/kg administered subcutaneously), doxycycline (5 mg/kg administeredorally), and enrofloxacin (5 mg/kg administered subcutaneously) and terbutaline (0.3 mg/kg administered orally), nebulisation (saline) and percussion, use of Ventolin 100 μg by puffer inhalation and terbutaline (0.3 mg/kg administered orally) as necessary to control dyspnoea. Prednisolone was given as a single daily dose of 2 mg/kg, and Seretide (Salmeterol 50 μg plus fluticasone 250 μg) was given using a mask and spacer. Response to treatment was marked, with improvement in respiratory effort and reduction in crackles audible on auscultation. This combination of treatment was maintained over the next month.

During this month of therapy the following observations were made:
1. There was a radiographic improvement characterised by 35 reduction in the prominence of the bronchointerstitial pattern and reduction in air entrapment.
2. Throughout each day, and from day to day, there was a marked variation in severity of respiratory clinical signs, although these were not as severe as those observed in the initial stages of the disease. Increase in respiratory effort was often observed when the cub was taken into the airconditioned nursery, during exercise or stress, and spontaneously, presumably in response to respiratory irritants in the environment.
3. Respiratory clinical signs responded rapidly to bronchodilators, given either orally terbutaline (0.3 mg/kg orally) or by inhalation (Ventolin puffer 100 μg).
4. Although response to therapy was marked, there were never times when the breathing pattern was normal.
5. The cub developed a poor “staring” coat, poor muscling, retarded growth rate, reduced activity level and playfulness, and relatively poor appetite when compared with her littermate.

The clinical findings and response to various therapies were reviewed, and the diagnosis of feline asthma was made. This decision was based upon the marked reactivity of the airways, marked and rapid response to bronchodilators, and general improvement of respiratory clinical signs with corticosteroid therapy.

Three months after onset of clinical signs, despite relatively aggressive oral corticosteroid therapy Prednisolone 2 mg/kg once daily, the level of control of the disease was stable but not yet satisfactory in terms of long-term health management. Consequently an experimental technique involving direct injection of Prednisolone sodium succinate, 1200 mg in a total volume of 30 ml (40 mg/ml aqueous solution), was instilled into the trachea under general anaesthesia. Recovery from the anaesthesia was uneventful, and within 24 hours there was a rapid and marked improvement in respiratory effort.

Kaasha's keepers unanimously reported that the breathing pattern improved to virtually normal levels for one week, with no episodes of dyspnoea during that period. However, the clinical pattern of laboured breathing returned to pre-treatment levels at seven to nine days post treatment. At this time therapy included oral corticosteroid at 2 mg/kg, use of inhaled Flixotide (Fluticasone 250 μg/dose) with and without Seretide (Salmeterol 50 μg plus fluticasone 250 μg/dose), use of Ventolin puffer, 100 μg, as necessary to control dyspnoea, and occasional use of Pulmicort nebules (Budesonide 400 μg) by nebulisation.

It is important to note that use of inhaled medications in the cub was characterised by variability in the effectiveness of the daily dose given, as a result of variation in her compliance, keeper compliance, keeper competence, and daily frequency of administration.

Three weeks after the intra-airway steroid procedure, another experimental therapy was applied. Injections of the C5a complement receptor antagonist AcF-[OPdChaWR] (1) were administered at a dose rate of 0.3 mg/kg as single daily subcutaneous injections for six days, followed by twice-weekly injections for 8 weeks. At this time the dose of oral corticosteriod, Prednisolone approx 1 mg/kg reducing, had been reduced to 20 mg per day because of concern regarding side effects of prolonged high dose use. Kaasha's keepers were asked to pay particular attention to ensuring that inhaled medications were being applied in the most effective manner, to compensate for reduction in the oral corticosteroid dose.

There was unanimous agreement among Kaasha's keepers that there was a moderate to marked improvement in breathing and in recovery time after episodes of dyspneoa following the week of daily AcF-[OPdChaWR] (1) injections. However, it was observed that the general breathing pattern was not as good during twice weekly treatments as it had been following daily injections, although recovery time was comparable.

The reduction in oral cortisone dosage and treatment with AcF-[OPdChaWR] (1) corresponded with a marked improvement in the playfulness, general activity level, appetite and general demeanour of the tiger cub. The addition of a further medication, Singulaire, was not associated with a noticeable improvement in clinical signs.

As at July 2002 Kaasha was approximately 32 kg and 7 months of age, and was being maintained on the following regimen:
Macrolone (prednisolone): 20 mg orally each evening; Singulaire (montelukast sodium): 10 mg orally each evening;
AcF-[OPdChaWR] (1): 3 mg/10 kg subcutaneously twice a week; Seretide puffer (Salmeterol 50 μg plus fluticasone 250 μg/dose:) morning and night, preceded by Ventolin, 100 μg, puffer;
Flixotide puffer (Fluticasone): 250 μg/dose up to four times each day; and
Pulmicort nebulisation (Budesonide): 400 μg once a day as time permits.

Further improvements in the therapeutic regime were tested with a view to long-term control of the asthma. Measures were taken to improve the efficiency of puffer medication delivery in an attempt to reduce or eliminate the oral corticosteroid use. At this time Kaasha showed mild dyspnoea at rest, and moderate to marked dyspnoea after exercise, exertion or stress, but this dyspnoea was not associated with visible distress. Her behaviour, growth rate and appetite were only slightly less than, or comparable with, her those of littermate.

A dietary trial was attempted, with complete replacement of the current diet by a different protein source, namely either lamb/mutton or rabbit exclusively.

Kaasha's condition deteriorated, and in early September 2002 she died under anaesthetic while undergoing a brain scan. From the pathology reports it appears that the hyperoesinophilia was affecting other organs apart from the lung and the animal was becoming increasingly ill from intestinal and renal effects. While it is not possible to draw any causal connection, it is noted that in August 2002 treatment with PMX53 had been discontinued. Post-mortem examination showed that the tiger cub had hyperoesinophilia syndrome, which contributed to the asthma-like lung condition, and also caused lesions in the kidney and intestine. This condition also occurs in humans.

### EXAMPLE 3

### Allergic Dermatitis in a Dog

A kelpie dog was treated with PMX53 (1 mg/kg/day PO) for intermittent lameness, which was noticeable after prolonged exercise. Because of the intermittent nature of the lameness the owner, a veterinarian, found it difficult to assess any improvement. However, the owner reported that the drug effected a marked improvement in the dog's allergic dermatitis, which had apparently resolved completely.

### EXAMPLE 4

### Treatment of Allergic Dermatitis in Dogs

Two dogs with dermatitis were treated with PMX53 (0.3 mg/kg in 30% polyethylene glycol 400: 70% 0.9% saline) as a subcutaneous injection once daily. Blood samples were collected after 4 weeks of treatment. One dog was then treated with 0.6 mg/kg PMX53 subcutaneously for 4 days before euthanasia and autopsy. Biochemistry and haematology was repeated on the high-dose dog at this time. No abnormalities were detected in the laboratory samples or on gross examination of the carcass. There was no evidence of irritation at the site of injection.

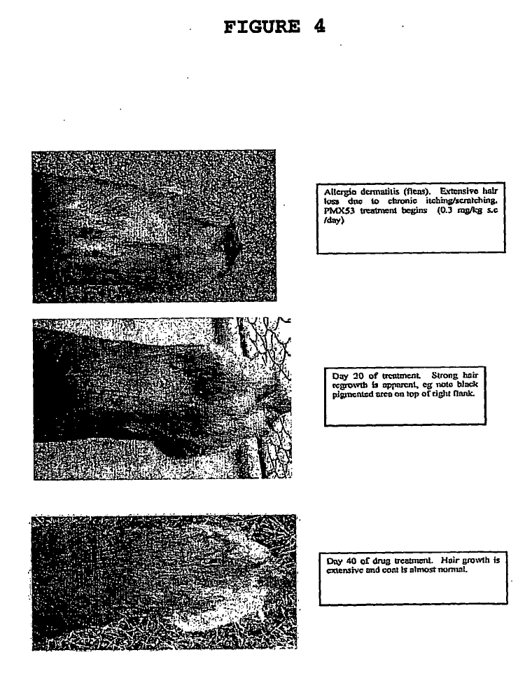

The second dog was bled for haematology and biochemistry after a total of seven weeks treatment. No abnormalities were detected. This dog had severe allergic dermatitis, which was presumed to be due to flea allergy; however, no antigen testing to confirm this was performed. The dermatitis completely resolved following treatment with PMX53, as shown in FIG. 4. Both dogs were healthy for the duration of the experiment, with no signs of drug toxicity.

A very old dog (estimated age 13-16 years) admitted to a pound was diagnosed as having severe atopic dermatitis affecting 100% of the skin and the inside of the ear pinnae (otitis external, and keratoconjunctivitis sicca (“dry eye”). The dog had broken skin over the dorsum of the tail, and both eyes were encrusted with yellow exudate.

Treatment of the skin condition with PMX53 was commenced using a topical preparation (5 mg/ml in 50% propylene glycol:50% water) applied to 25% of the body, including the tail, rump and right hind leg, once a day. The eyes were treated with PMX53 in an eye-drop formulation (5 mg/ml in 30% polyethylene glycol:70% normal saline). The sores on the tail resolved within 3 days. The thickening of the skin over the stifle and especially over the ischial tuberosity resolved noticeably, and the eyes improved to the point of being essentially normal in appearance. The dog showed no signs of itching.

The dog initially walked with a very stilted gait, but after treatment with PMX53 was able to walk and trot freely. This may either be due to an improvement in preexisting arthritis or to a less painful skin.

### EXAMPLE 5

### Treatment of Flea Allergy Dermatitis and Demodectic Dermatitis in Dogs

Demodex, also known as demodectic mange or red mange, is an infestation of the skin caused by the mite Demodex canis which causes dermatitis, skin thickening and hair loss, and is very common in dogs. This condition is thought to be due partially to impaired immune responses in the host. It is often associated with flea infestation, which itself can cause an allergic dermatitis. The skin irritation in infected animals is sometimes very extensive, and results in loss of hairs and severe skin rashes.

Given the infectious nature of Demodex canis infestation, corticosteroids are not a suitable treatment because they suppress local immune responses and worsen the condition by allowing the mites to proliferate.

(a) Two dogs suffering from demodectic dermatitis were treated for 13 days with PMX53 (0.3 mg/kg in 30% polyethylene glycol 400:70% 0.9% saline) by subcutaneous injection. No discomfort was noted on injecting this preparation. Both dogs showed a significant reduction in the inflammatory response in the skin, despite the fact that the challenge agent, fleas and demodex, had not been removed.

(b) A mastiff pup (approximately 6 months old) was diagnosed as having demodex infestation (folliculitis) of the head, involving both eyelids. This resulted in swelling of the lids, inversion of the lid margin and rubbing of hairs on the cornea (trichiasis). The eyes were red and discharging, and the dog squinted because of the ocular pain.

The skin lesions on the top of the head were treated daily with topical PMX53 daily (10 mg/ml in 30% polyethylene glycol: 70% 0.9% saline. This was applied to the lesion so that the lesion was wet; the volume to achieve this was not recorded. After 5 days of treatment the inflammation in the skin was reduced, although the mites were still present in scrapings taken from the lesion. This indicated that the drug can moderate inflammation associated with this condition without actually killing the parasite.

The eyelids were treated once daily with PMX53 eye drops (10 mg/ml in 30% polyethylene glycol:70% 0.9% saline. This was applied to the eyelid lesion so that the lesion was wet, and was also instilled into the eyes. Over 5 days the inflammation resolved to the point that the trichiasis was relieved and the dog's eyes were comfortable and functional. This was considered to be a very significant clinical response.

These results indicate that PMX53 may offer a means of controlling inflammation associated with the mite infestation without impairing immune responses which are required to eliminate the parasite. This demonstrates that PMX53 is a suitable anti-inflammatory agent to use where an infectious agent is present, and where common veterinary treatments such as glucocorticoids would be contraindicated because of their suppression of local immune responses.

### EXAMPLE 6

### Treatment of Asthma in Cats

Asthma in humans has many causes, including allergens, physical stimulants such as cold air or sulphur dioxide, and immune-based aetiologies. Both cats and horses have a recognised clinical condition which resembles human asthma. In horses with the condition known as “heaves”, asthma-like symptoms are caused by inhaled allergens, analogously to “allergic asthma”. In cats the cause of the airway inflammation can be uncertain, but its clinical signs resemble those seen in humans, with bronchoconstriction causing difficult breathing.

Cats provide a preferred clinical model of human disease for testing of the C5a antagonist of the invention, because PMX53 has been shown to bind well to the feline C5a receptor. PMX53 binds less effectively to the equine receptor, and the large size of horses means that administration of large quantities of drug is required. However, the equine model is not excluded.

Cats showing asthma-like respiratory pathology are selected from animals presented to veterinary practices. The diagnosis is confirmed by standard evaluation criteria, including routine blood biochemistry and haematology, chest X-ray and bronchoalveolar lavage. Cats are treated with PMX53 orally at a dose of 1 mg/kg or subcutaneously at 0.3 mg/kg. Response to treatment is evaluated using clinical parameters, such as easier breathing and reduction in peripheral blood eosinophilia. A repeat of the bronchoalveolar lavage to confirm a reduction in airway inflammation is also desirable.

Other animal model systems for asthma, in which asthma-like symptoms are provoked by defined stimuli, are known in the art, and may also be used in pre-clinical testing of the compounds of the invention. For example a sheep model is described in PCT/AU02/00715. A number of reviews have been published; see for example Tobin, 2003; Isenberg-Feig et al, 2003; Bice et al, 2000; Drazen et al, 1999; and http://ajrccm.atsjournals.org/cgi/collection/asthma_airway_animalmodels.

Discussion

Cyclic peptides have several important advantages over acyclic peptides as drug candidates (Fairlie et al 1995, Fairlie et al, 1998, Tyndall and Fairlie, 2001). The cyclic compounds described in this specification are stable to proteolytic degradation for at least several hours at 37° C. in human blood or plasma, in human or rat gastric juices, or in the presence of digestive enzymes such as pepsin, trypsin and chymotrypsin. In contrast, short linear peptides composed of L-amino acids are rapidly degraded to their component amino acids within a few minutes under these conditions. A second advantage lies in the constrained single conformations adopted by the cyclic and non-peptidic molecules, in contrast to acyclic or linear peptides, which are flexible enough to adopt multiple structures in solution other than the one required for receptor-binding. Thirdly, cyclic compounds such as those described in this invention are usually more lipid-soluble and more pharmacologically bioavailable as drugs than acyclic peptides, which can rarely be administered orally. Fourthly, the plasma half-lives of cyclic molecules are usually longer than those of peptides.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### REFERENCES

Bice D. E., Seagrave J, Green F. H. Inhal Toxicol. September 2000;12(9):829-62.
Drazen et al, Clin Exp Allergy 1999; 29 Suppl 2:37-47.
Fairlie, D. P., Wong, A. K.; West, M. W. Curr. Med. Chem., 1998, 5, 29-62.
Fairlie, D. P., Abbenante, G., and March, D. Curr. Med. Chem., 1995 2 672-705.
Gerard, C and Gerard, N. P. Ann. Rev. Immunol., 1994 12 775-808.
Isenberg-Feig H., Justice, J. P., Keane-Myers, A. Current Allergy and Asthma Reports 2003 3:70-78.
Konteatis, Z. D., Siciliano, S. J., Van Riper, G., Molineaux, C. J., Pandya, S., Fischer, P., Rosen, H., Mumford, R. A., and Springer, M. S. J. Immunol., 1994 153 4200-4204.
Sanderson, S. D., Kirnarsky, L., Sherman, S. A., Vogen, S. M., Prakesh, O., Ember, J. A., Finch, A. M. and Taylor, S. M. J. Med. Chem., 1995 38 3669-3675.
Strachan, A. J., Haaima, G., Fairlie, D. P. and Taylor, S. M. J Immunol. 164: 6560-6565, 2000.
Tobin M. J. Am. J. Respir. Crit. Care Med. 167 (3): 319
Tyndall, J. D. A. and Fairlie, D. P. Curr. Med. Chem. 2001, 8, 893-907.

1. A method of treatment of a hypersensitivity condition, comprising the step of administering an effective amount of an inhibitor of a G protein-coupled receptor to a subject in need of such treatment in which the inhibitor is a compound which
(a) is an antagonist of a G protein-coupled receptor,
(b) has substantially no agonist activity, and
(c) is a cyclic peptide or peptidomimetic compound of formula I 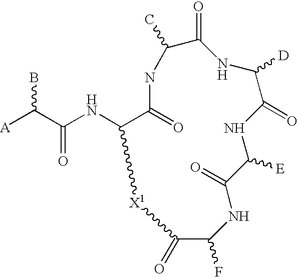
where A is H, alkyl, aryl, NH₂, NH-alkyl, N(alkyl)₂, NH-aryl, NH-acyl, NH-benzoyl, NHSO₃, NHSO₂-alkyl, NHSO₂-aryl, OH, O-alkyl, or O-aryl;
B is an alkyl, aryl, phenyl, benzyl, naphthyl or indole group, or the side chain of a D- or L-amino acid, but is not the side chain of glycine, D-phenylalanine, L-homophenylalanine, L-tryptophan, L-homotryptophan, L-tyrosine, or L-homotyrosine;
C is the side chain of a D-, L- or homo-amino acid, but is not the side chain of isoleucine, phenylalanine, or cyclohexylalanine;
D is the side chain of a neutral D-amino acid, but is not the side chain of glycine or D-alanine, a bulky planar side chain, or a bulky charged side chain;
E is a bulky substituent, but is not the side chain of D-tryptophan, L-N-methyltryptophan, L-homophenylalanine, L-2-naphthyl L-etrahydroisoquinoline, L-cyclohexylalanine, D-leucine, L-fluorenylalanine, or L-histidine;
F is the side chain of L-arginine, L-homoarginine, L-citrulline, or L-canavanine, or a bioisostere thereof; and
X is —(CH₂)ₙNNH— or (CH₂)ₙ—S—, where n is an integer of from 1 to 4; —(CH₂)₂O—; —(CH₂)₃O—; —(CH₂)₃—; —(CH₂)₄—; —CH₂COCHRNH—; or —CH₂—CHCOCHRNH—, where R is the side chain of any common or uncommon amino acid.

2. A method according to claim 1, in which n is 2 or 3.

3. A method according to claim 1, in which A is an acetamide group, an aminomethyl group, or a substituted or unsubstituted sulphonamide group.

4. A method according to claim 2, in which A is a substituted sulphonamide, and the substituent is an alkyl chain of 1 to 6 carbon atoms, or a phenyl or toluyl group.

5. A method according to claim 4, in which the substituent is an alkyl chain of 1 to 4 carbon atoms.

6. A method according to claim 1, in which B is the side chain of L-phenylalanine or L-phenylglycine.

7. A method according to claim 1, in which C is the side chain of glycine, alanine, leucine, valine, proline, hydroxyproline, or thioproline.

8. A method according to claim 1, in which D is the side chain of D-Leucine, D-homoleucine, D-cyclohexylalanine, D-homocyclohexylalanine, D-valine, D-norleucine, D-homo-norleucine, D-phenylalanine, D-tetrahydroisoquinoline, D-glutamine, D-glutamate, or D-tyrosine.

9. A method according to claim 1, in which E is the side chain of an amino acid selected from the group consisting of L-phenylalanine, L-tryptophan and L-homotryptophan, or is L-1-napthyl or L-3-benzothienyl alanine.

10. A method according to claim 1, in which the inhibitor is a compound which has antagonist activity against C5aR, and has no C5a agonist activity.

11. A method according to claim 1, in which the inhibitor has potent antagonist activity at sub-micromolar concentrations.

12. A method according to claim 1, in which the compound has a receptor affinity IC50<25 μM, and an antagonist potency IC50<1 μM.

13. A method according to claim 1, in which the compound is selected from the group consisting of compounds 1 to 6, 10 to 15, 17, 19, 20, 22, 25, 26, 28, 30, 31, 33 to 37, 39 to 45, 47 to 50, 52 to 58 and 60 to 70 described in PCT/AU02/01427.

14. A method according to claim 13, in which the compound is PMX53 (compound 1), compound 33, compound 60 or compound 45 described in PCT/AU02/01427.

15. A method according to claim 1, in which the inhibitor is used in conjunction with one or more other agents for the treatment of hypersensitivity conditions.

16. A method according to claim 15, in which the other agent is infliximab or is an inhibitor of C3a.

17. A method according to claim 1, in which the treatment is to prevent or alleviate acute recurrences of a hypersensitivity condition.

18. A method according to claim 1, in which the treatment is to prevent or alleviate a primary occurrence of a hypersensitivity condition.

19. A method according to claim 1, in which the hypersensitivity condition is selected from the group consisting of Type II immediate hypersensitivity (cytotoxic) and Type III (complex-mediated) immediate hypersensitivity, asthma, eczema, dermatitis, Arthus-type reactions, glomerulonephritis, hypereosinophilia syndrome, and farmer's lung.

20. A method according to claim 19, in which the hypersensitivity condition is eczema or dermatitis.

21. A method according to claim 20, in which the hypersensitivity condition is demodectic mange or flea allergy.

22. A method according to claim 20, in which the inhibitor is administered orally or topically.

23. A method according to claim 19, in which the hypersensitivity condition is asthma.

24. A method according to claim 22, in which the inhibitor is administered orally, intranasally or by inhalation.

25. A method according to claim 1, in which the inhibitor is used in conjunction with one or more other agents for the treatment of hypersensitivity conditions.
